(19) United States
(12) Patent Application Publication
(10) Pub. No.: US 2007/0053959 A1
(43) Pub. Date: Mar. 8, 2007

(54) Anti-perspirant/deodorant peel-on strip

(76) Inventors: Angelina W. Smith, Kosciusko, MS (US); Henderson Smith, Kosciusko, MS (US)

Correspondence Address: Galasso & Associates, LP, P.O. Box 26503, Austin, TX, 78755-0503, US

(21) Appl. No.: 11/513,936

(22) Filed: Aug. 31, 2006

Related U.S. Application Data
(60) Provisional application No. 60/713,997, filed on Sep. 2, 2005.

(51) Int. Cl. A61K 9/70; A61K 8/18

(52) U.S. Cl. 424/443; 424/65

(57) ABSTRACT

Anti-Perspirant/Deodorant Peel-On Strip is a strip with a protective film, adhesive layer, and a layer of deodorant and/or antiperspirant. The strip is ideally sealed inside a foil or other moisture resistant package. A user tears the top off of the package containing the strip and removes it from the package. Next, the consumer peels the protective film off of the strip to reveal the layer of deodorant and/or anti-perspirant. The consumer then uses the adhesive layer to apply the strip to the underarm. The strip is discarded after each use and a new strip can then be applied.

### CROSS REFERENCE TO RELATED APPLICATIONS

This Non-Provisional patent application claims priority to U.S. Provisional Patent Application No. 60/713,997 entitled “Anti Perspirant/Deodorant Peel-On Strip” filed on Sep. 2, 2005.

### FIELD OF THE DISCLOSURE

The disclosures made herein relate generally to the personal hygiene industry. The invention discussed herein is in the general classification of deodorants and anti-perspirants.

### BACKGROUND

The personal hygiene industry encompasses a variety of products, including shampoos, soaps, toothbrushes, toothpastes, creams and deodorants. Billions of dollars are spent each year on these products.

One of the products most often misused is deodorant. Many users are uncertain how to apply the proper amount of deodorant or anti-perspirant. When too little deodorant or anti-perspirant is applied, the results can be embarrassing. A malodorous smell or excessive perspiration can create problems in a personal relationship or business relationship. Conversely, application of an excessive amount of deodorant or antiperspirant is wasteful and can cause problems of its own. For example, the substances in the deodorant can cake onto and discolor clothing if an excessive amount of deodorant is utilized. Having an overpowering smell associated with excessive application of either deodorant or antiperspirant can also be embarrassing or undesirable.

A variety of aerosol sprays and deodorant sticks have been employed to fight perspiration and unpleasant body odors. However, these sprays and sticks are often bulky to transport and do not give the user a sense of the exact amount of product to apply.

Hence, there is a need in the art for an easy to use and inexpensive deodorant and anti-perspirant product that will be easy to transport and will permit a user to know exactly what quantity, of deodorant and/or antiperspirant to apply.

### SUMMARY OF THE DISCLOSURE

Anti-Perspirant/Deodorant Peel-On Strip is a deodorant and/or antiperspirant strip sealed in a moisture resistant package.

The principal object of this invention is to provide a deodorant and/or anti-perspirant strip that will permit a user to apply the appropriate amount of deodorant/anti-perspirant.

Another object of this invention is to provide an affordable deodorant and/or antiperspirant strip.

Another object of this invention is to provide an easy to transport deodorant and/or antiperspirant strip.

Another object of this invention is to provide a deodorant and/or anti-perspirant strip that will prevent messes and waste associated with traditional spray-on or stick-applied deodorant and/or antiperspirant.

Another object of this invention is to provide a deodorant and/or anti-perspirant strip having an adhesive strip for easy application.

Yet another object of this invention is to provide a device that effectively eliminates or reduces body odor and/or perspiration.

### BRIEF DESCRIPTION OF THE DRAWINGS

FIG. 1 depicts a perspective view of the preferred embodiment of the deodorant/anti-perspirant strip of the invention.

FIG. 2 depicts a perspective view of an alternative embodiment of the deodorant/anti-perspirant strip of the invention.

FIG. 3 depicts a perspective view of another alternative embodiment of the deodorant/anti-perspirant strip of the invention.

FIG. 4 depicts a perspective view of the preferred embodiment of the package containing the deodorant/anti-perspirant strip of the invention.

### DETAILED DESCRIPTION OF THE DRAWINGS

The preferred embodiment of Anti-Perspirant/Deodorant Peel-On Strip is comprised of a circular strip having a protective film over a layer of both deodorant and anti-perspirant on one side and an adhesive layer with an adhesive protective film on the opposite side. The circular strip is sealed inside a foil package.

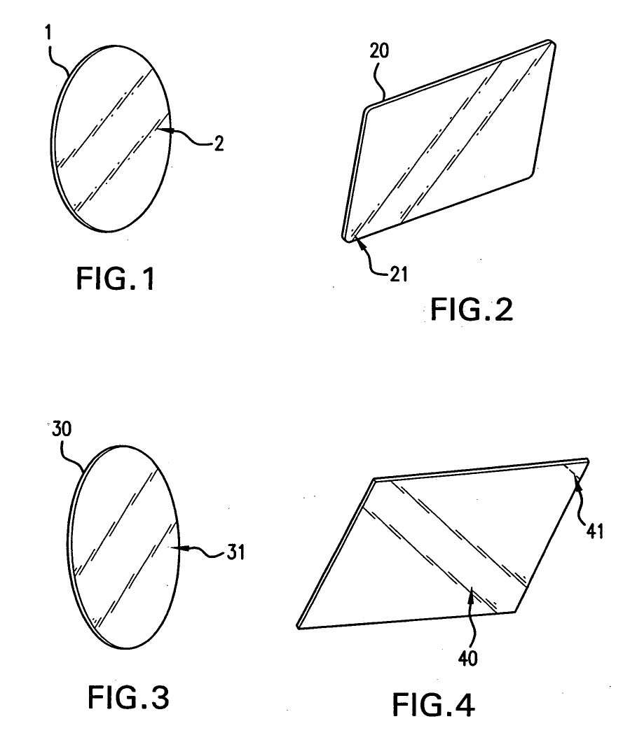

In the preferred embodiment of the invention, shown in FIG. 1, a layer of deodorant (not pictured) and a layer of antiperspirant (not pictured) evenly cover a circular strip 1. The top layer of deodorant or antiperspirant is covered by a circular protective film 2. The circular strip 1 and circular protective film 2 are approximately two inches in diameter. The combination of circular strip 1, layer of deodorant, layer of anti-perspirant and circular protective film 2 is approximately one-sixteenth of an inch thick. In this preferred embodiment, the circular strip 1 and the circular protective film 2 are made of plastic. In addition, a circular adhesive layer with an adhesive protective film is located on the back of the circular strip 1.

In another embodiment of the invention, shown in FIG. 2, a layer of deodorant (not pictured) and a layer of antiperspirant (not pictured) evenly cover a rectangular strip 20. The top layer of deodorant or antiperspirant is covered by a rectangular protective film 21. The rectangular strip 20 and rectangular protective film 21 are approximately two inches in length and one and a half inches in height. The combination of rectangular strip 20, layer of deodorant, layer of antiperspirant and rectangular protective film 21 is approximately one-sixteenth of an inch thick. In addition, a rectangular adhesive layer with an adhesive protective film is located on the back of the rectangular strip 20.

In another embodiment of the invention, shown in FIG. 3, a layer of deodorant (not pictured) and a layer of antiperspirant (not pictured) cover an oval strip 30. The top layer of deodorant or antiperspirant is covered by an oval protective film 31. The oval strip 30 and oval protective film 31 are approximately two inches in length and one and a half inches in width. Again, the combination of oval strip 30, layer of deodorant, layer of antiperspirant and oval protective film 31 is approximately one-sixteenth of an inch thick. In addition, an oval adhesive layer with an adhesive protective film is located on the back of the oval strip 30.

FIG. 4 shows the preferred embodiment of the packaging containing the strips of the present invention. A strip of the type described in FIGS. 1-3 is located within a foil package 40. The foil package 40 is rectangular in shape and dimensioned to be slightly larger than the strips of the invention. A peel corner 41, as is well known in the art, permits the front of the foil package to be easily removed. The interior of the package is lined with a nonstick poly substance to facilitate the removal of the strips. The foil package 40 is also designed to be moisture resistant.

In order to use Anti-Perspirant/Deodorant Peel-On Strip, a consumer tears the front off of the package containing the strip, utilizing the peel corner. The consumer then removes the strip from the package. Next, the consumer peels the protective film off of the strip to reveal the layer of deodorant and/or anti-perspirant. The consumer then removes the adhesive protective film to reveal, the adhesive layer which can be applied to the user's underarm. At the end of the day, the user can simply throw the strip into the garbage and use a new strip for the next application.

The components of Anti-Perspirant/Deodorant Peel-On Strip may vary widely but will likely include plastic and other components.

The plastic used in the production will ideally be selected for durability and longevity. Thermoplastics are commonly used in the manufacturing of components similar to those used in this invention. Polyethylene, polypropylene, and other similar thermoplastic materials would be among those with the necessary traits. Members of this family are recognized universally as being versatile and of high quality.

The plastic components of Anti-Perspirant/Deodorant Peel-On Strip could also be formed with the use of plastic molding techniques, such as injection molding or blow molding. Injection molding requires melted plastic to be forcefully injected into relatively cool molds. As the plastic begins to harden, it takes on the shape of the mold cavity. This technique is ideal for the mass production of products. Alternatively, blow molding, a form of extrusion, could be utilized. Blow molding involves a molten tube being pushed into a mold. Compressed air then forces the molten tube against the cold walls of the mold.

It should be obvious that the protective film, strips, packaging and other components of the present invention can be of various shapes and sizes.

It should also be obvious that the components of the invention can be made of different types of plastics or other suitable materials and can be of any color.

It will be recognized by those skilled in the art that changes or modifications may be made to the above-described embodiments without departing from the broad inventive concepts of the invention. It should therefore be understood that this invention is not limited to the particular embodiments described herein, but is intended to include all changes and modifications that are within the scope and spirit of the invention as set forth in the claims.

What is claimed is:

1. A personal hygiene device comprising a strip having a layer of deodorant.

2. The device of claim 1 wherein the strip is plastic.

3. The device of claim 1 wherein the strip is circular.

4. The device of claim 1 wherein the strip is rectangular.

5. The device of claim 1 wherein the strip is oval.

6. The device of claim 1 wherein the strip is square.

7. The device of claim 1 further comprising
a layer of antiperspirant.

8. The device of claim 1 further comprising
a protective film covering the layer of deodorant.

9. The device of claim 8 wherein the protective film is plastic

10. The device of claim 1 further comprising an adhesive layer located on the strip that is covered by an adhesive protective film.

11. The device of claim 1 further comprising
a package containing the strip.

12. The device of claim 11 wherein the package is foil.

13. The device of claim 11 wherein the package has a peel corner.

14. The device of claim 11 wherein the interior of the package is lined with a nonstick poly substance.

15. A personal hygiene device comprising:
(a) a strip that is plastic and circular covered with a layer of deodorant and a layer of antiperspirant on one side;
(b) a protective film that is plastic and circular that covers the layer of deodorant and the layer of anti-perspirant;
(c) an adhesive layer covered by an adhesive protective film located on the side of the strip opposite the layer of deodorant and the layer of anti-perspirant; and
(d) a foil package containing the strip and having a peel corner and an interior nonstick poly substance lining.
